(19) United States
(12) Patent Application Publication
(10) Pub. No.: US 2005/0124943 A1
(43) Pub. Date: Jun. 9, 2005

(54) Medical bi-directional in-out switchable irrigation-drainage system for intracranial surgery

(75) Inventor: Lin-Hsue Yang, Banciao City (TW)

Correspondence Address: BACON & THOMAS, PLLC, 625 SLATERS LANE, FOURTH FLOOR, ALEXANDRIA, VA, 22314, US

(73) Assignee: Lin-Hsue Yang, Banciao City (TW)

(21) Appl. No.: 11/041,235

(22) Filed: Jan. 25, 2005

(30) Foreign Application Priority Data
Oct. 21, 2003 (TW) 092129169

(51) Int. Cl. A61M 5/00

(52) U.S. Cl. 604/248

(57) ABSTRACT

A medical bi-directional in-out switchable irrigation-drainage system for intracranial surgery according to the present invention has a four-way adapter containing a knob having a rotatable interior baffle and four hollow connectors. The knob is assembled to the center of the four-way adapter. A passageway interconnects two hollow connectors next to each other and two separately isolated division passageways are formed by turning the knob. The system further has a pressure control bottle; a first connecting tube for connecting the pressure control bottle and the four-way adapter; a second connecting tube for connecting to a target tissue; and a third connecting tube for connecting to the other target tissue. The first, second and third connecting tubes are connected to different connectors of the four-way adapter respectively, and the rotatable interior baffle is actuated together with the knob.

### BACKGROUND OF THE INVENTION

1. Field of the Invention

The present invention relates to a medical bi-directional in-out switchable irrigation-drainage system, and more particularly to an irrigation-drainage system suited for an intracranial surgical operation which is able to make a bi-directional in-out switchable changing so as to irrigate with a liquid medicine or to drain a waste fluid off a tissue through a single tube.

2. Description of Related Art

In surgery, placement and administration of an irrigation-drainage tube will be very helpful in tissue healing. The irrigation-drainage tube is used to drain off the blood, secretions and fluids gathering in the dead space of wound cavity. Thus, the possibility of getting infection is decreased, furthermore, skin tissues surrounding a swelling wound are prevented from being stressed and irritated so that the wound can therefore be healed at an optimum rate. The irrigation-drainage tube can also be a pathway for liquid drug delivery to a target tissue. For example, the use of a drainage tube is subject to the hemostatic process during a surgical operation. If oozing continues, it is necessary to use the drainage tube to drain the waste blood off. In general, the drainage tube is categorized into a closed configuration for discharging a waste fluid in the wound cavity and avoiding contamination, and an open configuration capable of discharging fluids by rubber tissue and penrose drain, for example. The open configuration is the most widely used for discharging fluids. When the drainage tube is used, changes of the fluid being drawn off in the tube can be used to determine the treatment condition of a wound. In normal case, the color of the fluid will gradually change from maroon to pink, and also, the amount of the fluid being withdrawn will decrease. On the contrary, if the amount of the fluid being drawn off increases dramatically or the fluid color changes, it will be considered abnormal and a further treatment for the wound will be required.

In the case of cerebrovascular disease such as intracerebral hemorrhage which refers to the hemorrhagic stroke, such as thalamic hemorrhage, basal ganglia hemorrhage, subcortical hemorrhage, cerebellar hemorrhage and brainstem hemorrhage In treating such a intracerebral hemorrahge, the blood clots are usually removed by surgical operation or positioning a drainage tube to withdraw blood remaining in the tube or blood clots via the drainage tube. Or alternatively, a thrombolytic agent such as r-TPA and urokinase is infused within the blood clots to dissolve the blood clots after the surgical operation prior to the drainage. Conventionally, only a single catheter is used for irrigation and not much irrigation buffer can be infused in the meantime. Thus, the conventional catheter is not efficient and tends to clog easily. Otherwise, two catheters can be used for irrigation and drainage respectively for irrigating the residual blood stasis with a lot of irrigation buffer.

However, the conventional irrigation-drainage tube can perform only with a one-way switcher which has an inlet port and an outlet port. When a changeover of flow path is required, the connector of the drainage tube has to be disconnected and reconnected for an instant so that the fluid in the tube will be changed from a drainage flow of blood into an irrigation flow of liquid medicine. This action not only causes an inconvenience for operation during the surgery but also increases the risk of infection.

In addition, the conventional irrigation-drainage-like devices require separate pressure detecting modules for monitoring the pressure of the input catheter and output catheter respectively. This increases the cost which is obviously undesirable. The inventor is thus motivated to provide a switchable irrigation-drainage system for medical input/output changing to overcome the above-mentioned problems.

### SUMMARY OF THE INVENTION

A primary object of the present invention is to provide a changeover adapter for medical input/output and a medical bi-directional in-out switchable irrigation-drainage system for instantly changing over the flow path of an irrigation buffer or a drainage fluid during or after a surgical operation, thereby avoiding an inadvertent situation caused by the complicated disconnection and reconnection of a connector and reducing the possibility of infection. Also, the present invention operates in a two-way manner so as to reduce the possibility of having a blood clot during irrigation or drainage and to lengthen the catheter lifetime.

Another object of the present invention is to provide a changeover adapter for medical input/output and a medical bi-directional in-out switchable irrigation-drainage system which is able to connect an automatic switcher for changing over irrigation fluid so as to flush the tissues bi-directionally and repeatedly on schedule during or after a surgical operation, provide more flexible medical treatment, reduce the possibilities of bringing about contamination and clogging of the catheter, and avoid potential contamination problems caused by personal operation or from the risk of mis-connecting the catheter.

A further object of the present invention is to provide a changeover adapter for medical input/output and a medical bi-directional in-out switchable irrigation-drainage system to be used with only one pressure detector so as to detect the respective pressure within two tissues being connected simultaneously with the pressure detector and provide further medical information without an additional pressure detecting unit.

To attain the aforesaid objects, a changeover adapter for medical input/output according to the present invention constituted by a knob containing an interior baffle and four hollow connectors, where the knob is assembled to the center of the adapter in a four-way fashion. Thus, two passageways are formed, and each is composed with two interconnecting hollow connectors next to each other by turning the adapter.

A medical bi-directional in-out switchable irrigation-drainage system for intracranial surgery according to the present invention, which comprises a four-way adapter containing a knob having an interior baffle and four hollow connectors, where the knob is assembled to the center of the four-way adapter, and a passageway interconnecting two hollow connectors next to each other and two separately isolated division passageways are formed by turning the knob; a pressure control bottle for regulating the pressure used to withdraw the fluid in the tissues (namely, the irrigation buffer in the tissue will be drain off automatically when the pressure of the buffer reaches a predetermined level); a first connecting tube for connecting the pressure control bottle and the four-way adapter; a second connecting tube for connecting to a target tissue and one of the hollow connectors of the four-way adapter; and a third connecting tube for connecting to the other target tissue and one of the hollow connectors of the four-way adapter; wherein the first, second and third connecting tubes are connected to different connectors of the four-way adapter respectively, and the baffle in the knob is actuated together with the knob.

The material of a changeover adapter according to the present invention is not specifically defined. Preferably, the adapter is made of plastic, latex, silica gel or any other materials suited for medical purposes. The hollow connectors are not specifically defined, and preferably, are airtight sealed luer-lock connectors including at least two male connectors to maintain the seal of reconnection. The position of the respective connectors is not specifically defined. Preferably, the connectors are mounted in an opposed manner. It is preferable for the four-way adapter to further include a plurality of interior positioning detents for facilitating the position of the knob after assembly. The rotation angle of the knob is not specifically defined, and preferably, is set to 90°.

A medical bidirectional in-out switchable irrigation-drainage system for intracranial surgery according to the present invention further comprises a liquid medicine-supplying unit so as to provide a medication specifically for treating the tissues or dissolving a blood clot during or after a surgical operation to facilitate perfusion of the liquid medication. The present system employs gravity for the irrigation. In the optimum case, the irrigation buffer comes from a position 20-30 cm above the head of a patient. Thus, the difference in height between the irrigation buffer and the pressure bottle causes the infusion. Once the outlet port is clogged as a result of the intracranial pressure for example, it can be noticed that the flow rate of the irrigation buffer will gradually slow and finally stagnate, without causing over-perfusion damage. Any pathological disorders can also be found for timely treatment. Meanwhile, the body fluid or blood still can be withdrawn by drainage at the same time.

The pressure control bottle of the present invention is not specifically defined. Preferably, it is a plastic bottle with graduation. Further, it comprises a fifth connecting tube and a liquid collection unit for receiving body fluids or a waste liquid. The liquid collection unit according to the present invention is not specifically defined, and preferably, is a sealed bag or bottle. The present device can further comprise a plurality of automatic switching valves having positioning detents mounted on the first connecting tube, the second connecting tube and the third connecting tube respectively for facilitating control of the opening angle of the valves in use so as to set the flow rate of every perfusion or drainage.

A detecting unit for medical-related purposes can be accompanied with a medical bi-directional in-out switchable irrigation-drainage system according to the present invention. For example, a pressure detecting chip is included to instantly measure the pressure in an enclosed tissue for determining the suitable way for treatment.

Other objects, advantages, and novel features of the invention will become more apparent from the following detailed description when taken in conjunction with the accompanying drawings.

### BRIEF DESCRIPTION OF THE DRAWINGS

FIG. 1 is an expanded perspective view of a four-way changeover adapter for medical input/output according to the present invention.

FIGS. 2a and 2b are cross-sectional views of division passageways in a four-way changeover adapter for medical input/output according to the present invention.

FIG. 3 is a schematic view of a medical bi-directional in-out switchable irrigation-drainage system according to a second embodiment of the present invention.

FIG. 4 is a schematic view of a medical bi-directional in-out switchable irrigation-drainage system according to a third embodiment of the present invention.

### DETAILED DESCRIPTION OF THE PREFERRED EMBODIMENT

Three preferred embodiments of the present invention will be described below with reference to drawings for illustrating the technical features of this invention.

Embodiment 1

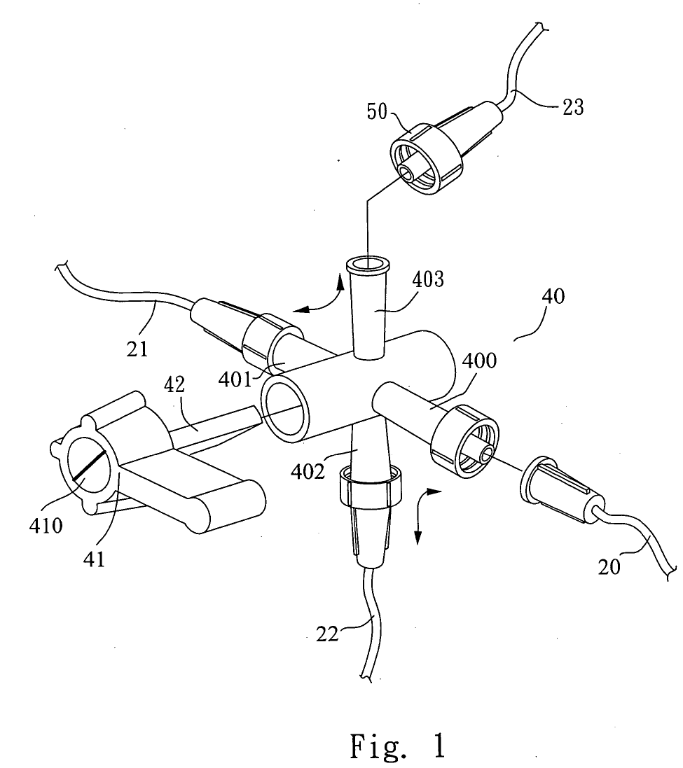

Referring to FIG. 1, a changeover adapter 40 for medical input/output according to the present invention comprises a knob 41 having an interior baffle 42 and four hollow connectors 400, 401, 402, 403, where the four hollow connectors are composed of two male connectors 402, 403 arranged in opposition to each other and two female connectors 400, 401 arranged in opposition to each other so as to form a four-way adapter 40 by interconnection thereof. The knob 41 is assembled to the center of the four-way adapter 40 so that a passageway interconnecting two hollow connectors next to each other is formed by turning the knob 41 and that two separately isolated division passageways are formed in the four-way adapter 40 by means of the baffle 42, as exemplified by both the connection of the hollow connector 400 to the hollow connector 403 and the connection of the hollow connector 401 to the hollow connector 402.

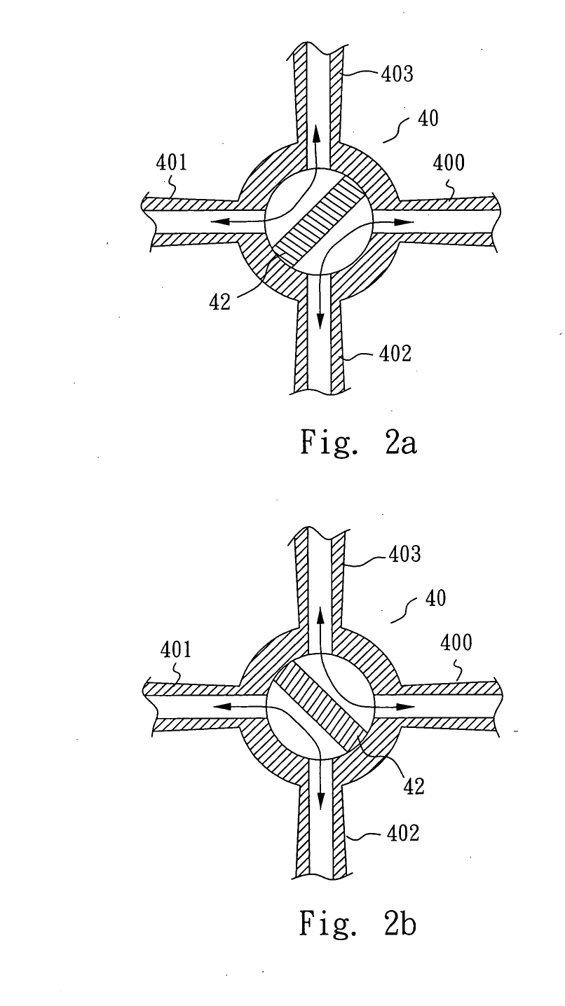

Referring to FIG. 2 which illustrates a schematic view of the division passageways, when the baffle 42 in the knob 41 of the changeover adapter 40 for a medical irrigation-drainage system according to the present invention is positioned as shown in FIG. 2a, a division passageway is formed by interconnecting the hollow connectors 403 and 401 while the other division passageway is formed by interconnecting the hollow connectors 400 and 402. When the baffle 42 in the knob 41 is positioned as shown in FIG. 2b, a division passageway is formed by interconnecting the hollow connectors 403 and 400 while the other division passageway is formed by interconnecting the hollow connectors 401 and 402. In this embodiment, the hollow connectors are luer-lock connectors to maintain the seal of the reconnection. The four-way adapter of the present invention further comprises a plurality of interior positioning detents (not shown) for facilitating position of the knob after assembly and restricting the rotation angle of the knob.

Embodiment 2

In addition to the changeover adapter for medical input/output as provided by the present invention, a bi-directional in-out switchable irrigation-drainage system is developed.

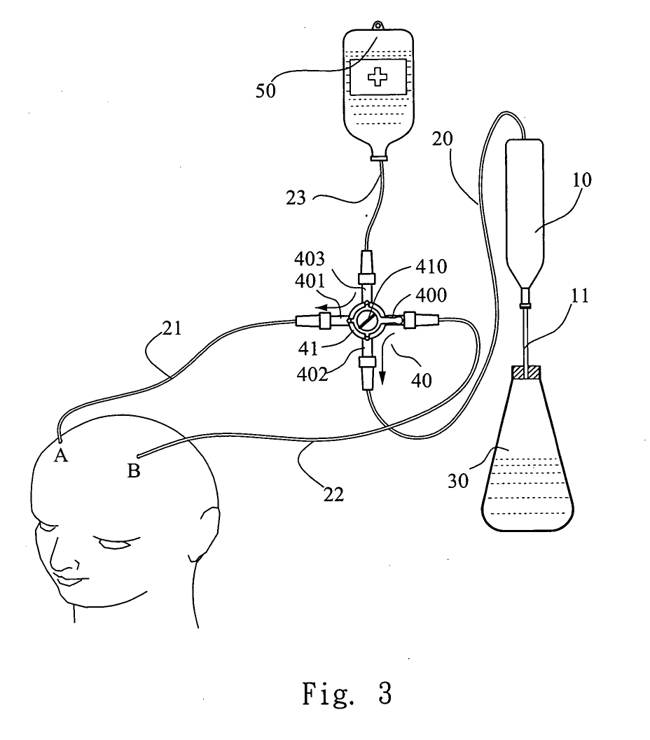

Referring to FIG. 3, in this embodiment, the pressure control bottle is connected to the first connecting tube 20 at the beginning. Then, the other end of the first connecting tube 20 is connected to a connector 402 of the four-way adapter 40. Also, the connector 400 is connected to the third connecting tube 22; the connector 401 is connected to the second connecting tube 21; and the connector 403 is connected to the fourth connecting tube 23. The connectors used in this embodiment are luer-lock connectors (denoted as 50 in FIG. 1). The knob 41 mounted at the center of the four-way adapter 40 is marked with a graduation line or indention 410 from the up-right side to the bottom-left side as those marked in the first embodiment. The flow path of the irrigation-drainage fluid of this system is directed as follows. A medicine 50 reaches tissue A by flowing through the fourth connecting tube 23, the connector 403, the connector 401 and the second connecting tube 21 while body fluid of tissue B enters the pressure control bottle 10 by flowing through the third connecting tube 22, the connector 400, the connector 402 and the first connecting tube 20; being ultimately ended in a liquid collection unit 30 through a fifth connecting tube 11.

Embodiment 3

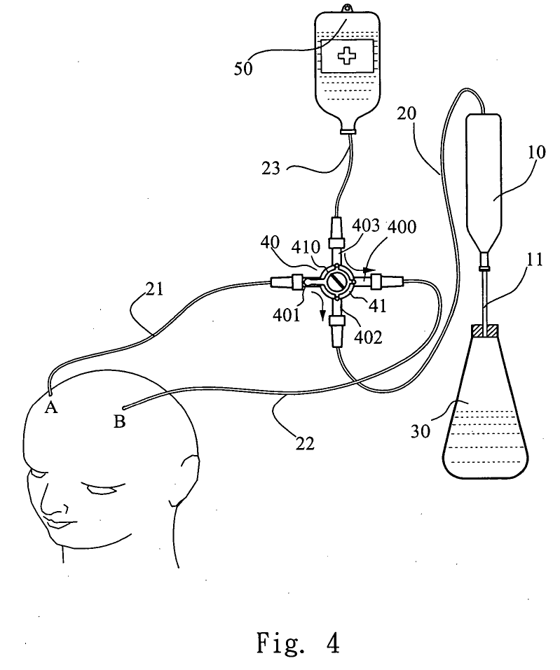

Referring now to FIG. 4, the flow path of this embodiment is contrary to that of the second embodiment. Even so, the components and interconnections of this embodiment are the same as those of the second embodiment, except for the graduation line or indention 410 marked on the knob 41 is otherwise arranged from the up-left side to the bottom-right side for switching the knob 41. Accordingly, the flow path of the irrigation-drainage fluid of this system is directed as follows. The medication 50 reaches tissue B by flowing through the fourth connecting tube 23, the connector 403, the connector 400 and the third connecting tube 22 while the body fluid of tissue A enters the pressure control bottle 10 by flowing through the second connecting tube 21, the connector 401, the connector 402 and the first connecting tube 20; being ultimately ended in the liquid collection unit 30 through the fifth connecting tube 11.

The second and the third embodiments can further connect an automatic switch for changeover on schedule to control the knob 41 at the center of the four-way adapter 40 so that the knob 41 of the irrigation-drainage system will automatically switch to a position at a predetermined point of time. Hence, the knob can be switched repeatedly to flush the tissues reciprocally. The flow rate for removing the blood clots in the tissue by the flush is thus accelerated. Also, clogging in the respective perfusion tube as a result of long-time one-way fluid flow is avoided, and the system is more flexible than prior art devices. Potential contamination problems caused by personal operation or the risk of reconnecting the tube by mistake are also prevented.

During a surgical operation, especially intracranial surgery, control of the patient's intracranial pressure is most critical of all. Due to the two-way device of the present invention, the flow path can be switched instantly or on schedule to detect the pressure in the two tissues being connected without additionally connecting to any other pressure detecting unit. Furthermore, a pressure detecting chip can be further included to measure the pressure in a tissue during or after a surgical operation to control the course of treatment.

Although the present invention has been explained in relation to its preferred embodiments, it is to be understood that many other possible modifications and variations can be made without departing from the spirit and scope of the invention as hereinafter claimed.

1. A medical bi-directional in-out switchable irrigation-drainage system for intracranial surgery, comprising:
a four-way adapter containing a knob having an interior baffle and four hollow connectors, wherein said knob is assembled to the center of said adapter in a four-way fashion, and a passageway interconnecting two of said hollow connectors next to each other and two separately isolated division passageways are formed by turning the knob;
a pressure control bottle for regulating the pressure of said system;
a first connecting tube for connecting to said pressure control bottle and said four-way adapter;
a second connecting tube for connecting to a target tissue and one of said hollow connectors of said four-way adapter; and
a third connecting tube for connecting to the other target tissue and one of said hollow connectors of said four-way adapter;
wherein said first, second and third connecting tubes are connected to different connectors of said four-way adapter respectively, and said interior baffle is rotatable and it was actuated together with said knob.

2. The system of claim 1, further comprising a medicine supplying unit and a fourth connecting tube through which said medicine supplying unit is communicated with said four-way adapter.

3. The system of claim 1, wherein said four-way adapter has at least two male luer-lock connectors.

4. The system of claim 1, wherein a plurality of positioning detents are interiorly disposed at the interconnecting position in said four-way adapter for facilitating position of said knob after assembly.

5. The system of claim 1, wherein said pressure control bottle further comprises a fifth connecting tube for connection and a liquid collection unit for receiving body fluids or a waste fluid.

6. The system of claim 1, further comprising a plurality of automatic switching valves mounted on said first connecting tube, said second connecting tube and said third connecting tube respectively.

7. The system of claim 1, further comprising a pressure detecting unit.

8. The system of claim 7, wherein said pressure detecting unit is a pressure detecting chip.

9. The system of claim 1, further comprising an automatic switching unit for changeover.

10. The system of claim 9, wherein said automatic switching unit for changeover is a time switch.
